(19) United States
(12) Patent Application Publication
(10) Pub. No.: US 2022/0016448 A1
(43) Pub. Date: Jan. 20, 2022

(54) REMOVABLE MASK AIRFLOW DEVICE

(71) Applicant: MAKE GREAT SALES LIMITED, Wanchai (HK)

(72) Inventors: Bruce Peter Cran, Ho Chi Minh City (VN); Kin Choi Lee, Wanchai (HK)

(21) Appl. No.: 17/193,109

(22) Filed: Mar. 5, 2021

Related U.S. Application Data
(63) Continuation-in-part of application No. 29/749,381, filed on Sep. 4, 2020.
Application No. 29/749,381 is a continuation-in-part of application No. 29/754,173, filed on Oct. 7, 2020.
(60) Provisional application No. 63/053,813, filed on Jul. 20, 2020.

(51) Int. Cl. A62B 18/00; A61L 9/20; A62B 18/08; A62B 7/10; A62B 9/00; F04D 29/70; F04D 27/00; B01D 46/00

(52) U.S. Cl. CPC A62B 18/006; A61L 9/20; A62B 18/08; A62B 7/10; A62B 9/00; A62B 23/02; F04D 27/004; B01D 46/0028; A61L 2209/14; A61L 2209/11; F04D 29/701

(57) ABSTRACT

A removable mask airflow device, comprising a first portion constructed and arranged to be positioned at an exterior surface of a facemask, the first portion comprising an opening and at least one flow processing device disposed at the opening and a second portion constructed and arranged to be positioned at an interior surface of the facemask. The at least one flow processing device is configured to process fluid flow through the opening. The second portion is constructed and arranged to couple with the first portion with a portion of the facemask being between the first portion and the second portion.

### CROSS REFERENCE TO RELATED APPLICATIONS

This application claims benefit of U.S. Provisional Application No. 63/053,813, filed Jul. 20, 2020, and entitled REMOVABLE AIR REGULATOR DEVICE, which is hereby incorporated by reference in its entirety.

This application claims benefit of U.S. Design Application No. 29/749,381, filed Sep. 4, 2020, and entitled MASK FILTER, which is hereby incorporated by reference in its entirety.

This application claims benefit of U.S. Design Application No. 29/754,173, filed Oct. 7, 2020, and entitled REMOVABLE AIR REGULATOR DEVICE, which is hereby incorporated by reference in its entirety.

### FIELD OF INTEREST

The present inventive concepts relate to the field of personal use facemasks, and more particularly to devices for regulating and/or filtering respiratory flow via a facemask.

### BACKGROUND

There are many circumstances in which an individual may need to control and/or regulate the respiratory flow of nearby gas/aerosol. For example, in some circumstances a user may wear a facemask to prevent exposure to fumes, viruses, bacteria, airborne contaminants, etc., collectively “material.” In some circumstances, to address such concerns, the individual may select a mask comprising one or more fabric or fabric-like layers designed to filter the unwanted material. However, in some circumstances, wearing such a mask may result in other problems, such as difficulty breathing or general lack of comfort. In other circumstances, such a mask may not be available or affordable.

It would be advantageous for the individual to have a mechanism for controlling and/or regulating the respiratory flow of nearby gas/aerosol flow to enhance personal comfort and safety. More particularly, it would be advantageous to have a flow control mechanism that can be removably coupled to a facemask, and reused with different facemasks.

### SUMMARY

In accordance with the inventive concepts, provided is a removable airflow device, comprising a first portion constructed and arranged to be positioned at an exterior surface of a facemask, the first portion comprising an opening and at least one flow processing device disposed in the opening, the at least one flow processing device configured to process fluid flow through the opening; and a second portion constructed and arranged to be positioned at an interior surface of the facemask. The second portion is constructed and arranged to couple with the first portion with a portion of the facemask being between the first portion and the second portion.

In various embodiments, the second portion is removably coupled to the first portion.

In various embodiments, the second portion includes at least one magnetic device configured to magnetically couple to the first portion.

In various embodiments, the at least one flow processing device comprises an ultraviolet light source, the ultraviolet light source oriented to degrade or destroy bacteria and/or viruses passing through the opening.

In various embodiments, the flow processing device comprises a fan disposed within or proximate the opening of the first portion.

In various embodiments, the fan is a variable speed fan.

In various embodiments, the flow processing device further comprises a filter disposed at the opening.

In various embodiments, the device further comprises at least one control mechanism and a power source configured to operatively control the at least one flow processing device.

In various embodiments, the at least one flow processing device is a plurality flow processing devices.

In various embodiments, the plurality flow processing devices comprises at least two different types of flow processing devices selected from a group consisting of a fan, a filter, and an ultraviolet light emitter.

In accordance with the inventive concepts, provided is a removable airflow device, comprising a first portion constructed and arranged to be positioned at an exterior surface of a facemask; a second portion constructed and arranged to be positioned at an interior surface of the facemask; and at least one flow processing device having an opening and disposed within the first and/or second portions, the at least one flow processing device configured to process fluid flow through the opening. The first and second portions form at least one clip are constructed and arranged to receive the facemask.

In various embodiments, the first portion comprises a first leg, a second leg, and a central portion between the first and second legs, wherein the central portion is configured to fit and/or conform to a user's nose.

In various embodiments, the first portion further comprises at least one flow processing device in the first or second leg, which is selected from a group consisting of a fan, a filter, and an ultraviolet light emitter.

In various embodiments, the at least one flow processing device comprises an ultraviolet light source, the ultraviolet light source oriented to degrade or destroy bacteria and/or viruses passing through the opening.

In various embodiments, the at least one flow processing further comprises a fan disposed within or proximate the opening.

In various embodiments, the fan is a variable speed fan.

In various embodiments, the at least one flow processing further comprises a filter disposed at the opening.

In various embodiments, the device further comprises at least one control mechanism and power source configured to operatively control the at least one flow processing device.

In various embodiments, the at least one flow processing device is a plurality flow processing devices, and the plurality flow processing devices comprises at least two different types of flow processing devices selected from a group consisting of a fan, a filter, and an ultraviolet light emitter.

In various embodiments, the first leg and the second leg each include at least one of the flow processing devices.

### BRIEF DESCRIPTION OF THE DRAWINGS

The present inventive concepts will become more apparent in view of the attached drawings and accompanying detailed description. The embodiments depicted therein are provided by way of example, not by way of limitation, wherein like reference numerals refer to the same or similar elements. The drawings are not necessarily to scale, emphasis instead being placed upon illustrating aspects of the invention. Below is a brief description of the drawings.

FIG. 1 is a front-perspective view of an embodiment of a removable mask airflow device, in accordance with aspects of inventive concepts.

FIG. 2 is a rear-perspective view of the removable mask airflow device of FIG. 1, in accordance with aspects of inventive concepts.

FIG. 3 is a front view of the removable mask airflow device of FIG. 1, in accordance with aspects of inventive concepts.

FIG. 4 is another front-perspective view of the removable mask airflow device of FIG. 1, in accordance with aspects of inventive concepts.

FIG. 5 is another rear-perspective view of the removable mask airflow device of FIG. 1, in accordance with aspects of inventive concepts.

FIG. 6 is another front-perspective view of the removable mask airflow device of FIG. 1, in accordance with aspects of inventive concepts.

FIG. 7 is a rear view of the removable mask airflow device of FIG. 1, in accordance with aspects of inventive concepts.

FIG. 8 is a partially unassembled, bottom view of the removable mask airflow device of FIG. 1, in accordance with aspects of inventive concepts.

FIG. 9 is a side view of the removable mask airflow device of FIG. 8, in accordance with aspects of inventive concepts.

FIG. 10 is a front-perspective, partially-exploded view of an embodiment of the removable mask airflow device of FIG. 1, in accordance with aspects of inventive concepts.

FIG. 11 is another front-perspective view of the removable mask airflow device of FIG. 11, in accordance with aspects of inventive concepts.

FIG. 12 shows a user wearing a facemask including the removable mask airflow device FIG. 1, in accordance with aspects of inventive concepts.

FIG. 13 is a rear-perspective view of the embodiment of the removable mask airflow device on a mask of FIG. 12, in accordance with aspects of inventive concepts.

FIG. 14 is a perspective view of an alternative embodiment of a removable mask airflow device, in accordance with aspects of inventive concepts.

FIG. 15 is a perspective view of the removable mask airflow device of FIG. 14, in accordance with aspects of inventive concepts.

FIG. 16 is a front view of the removable mask airflow device of FIG. 14, in accordance with aspects of inventive concepts.

FIG. 17 is a rear view of the removable mask airflow device of FIG. 14, in accordance with aspects of inventive concepts.

FIG. 18 is a first side view of the removable mask airflow device of FIG. 14, in accordance with aspects of inventive concepts.

FIG. 19 is a second side view of the removable mask airflow device of FIG. 14, in accordance with aspects of inventive concepts.

FIG. 20 is a bottom view of the removable mask airflow device of FIG. 14, in accordance with aspects of inventive concepts.

FIG. 21 is a top view of the removable mask airflow device of FIG. 14, in accordance with aspects of inventive concepts.

FIG. 22 is a front-perspective, partially-exploded view of another embodiment of a removable mask airflow device, in accordance with aspects of inventive concepts.

FIG. 23 shows a user wearing the removable mask airflow device of FIG. 22, in accordance with aspects of inventive concepts.

FIG. 24 is a front-perspective, partially unassembled view of another embodiment of a removable mask airflow device, in accordance with aspects of inventive concepts.

FIG. 25 is a front-perspective view of the removable mask airflow device of FIG. 24, in accordance with aspects of inventive concepts.

FIG. 26 is a top view of the removable mask airflow device of FIG. 24, in accordance with aspects of inventive concepts.

FIG. 27 is a top-perspective view of the removable mask airflow device of FIG. 24, in accordance with aspects of inventive concepts.

FIG. 28 is a front-perspective view of the removable mask airflow device of FIG. 24, in accordance with aspects of inventive concepts.

FIG. 29 is a side view of the removable mask airflow device of FIG. 24, in accordance with aspects of inventive concepts.

FIG. 30 is a rear view of the removable mask airflow device of FIG. 24, in accordance with aspects of inventive concepts.

FIG. 31 shows a user wearing the removable mask airflow device of FIG. 24 on a facemask, in accordance with aspects of inventive concepts.

FIG. 32 is a front-perspective view of another embodiment of a removable mask airflow device, in accordance with aspects of inventive concepts.

FIG. 33 shows a user wearing the removable mask airflow device of FIG. 32 on a mask, in accordance with aspects of inventive concepts.

FIG. 34 shows a perspective view of an embodiment of a round filter configured for a removable mask airflow device, in accordance with aspects of inventive concepts.

FIG. 35 shows a front view of the filter of FIG. 34, in accordance with aspects of inventive concepts.

FIG. 36 shows a rear view of the filter of FIG. 34, in accordance with aspects of inventive concepts.

FIG. 37 shows a side view of the filter of FIG. 34, in accordance with aspects of inventive concepts.

FIG. 38 shows a perspective view of an embodiment of a square filter configured for a removable mask airflow device, in accordance with aspects of inventive concepts.

FIG. 39 shows a front view of the filter of FIG. 38, in accordance with aspects of inventive concepts.

FIG. 40 shows a rear view of the filter of FIG. 38, in accordance with aspects of inventive concepts.

FIG. 41 shows a side view of the filter of FIG. 38, in accordance with aspects of inventive concepts.

FIG. 42 shows a perspective view of an embodiment of a rectangular filter configured for a removable mask airflow device, in accordance with aspects of inventive concepts.

FIG. 43 shows a front view of the filter of FIG. 42, in accordance with aspects of inventive concepts.

FIG. 44 shows a rear view of the filter of FIG. 42, in accordance with aspects of inventive concepts.

FIG. 45 shows a side view of the filter of FIG. 42, in accordance with aspects of inventive concepts.

FIG. 46 shows a perspective view of an octagon filter configured for a removable mask airflow device, in accordance with aspects of inventive concepts.

FIG. 47 shows a front view of the filter of FIG. 46, in accordance with aspects of inventive concepts.

FIG. 48 shows a rear view of the filter of FIG. 46, in accordance with aspects of inventive concepts.

FIG. 49 shows a side view of the filter of FIG. 46, in accordance with aspects of inventive concepts.

### DETAILED DESCRIPTION OF EXEMPLARY EMBODIMENTS

Various aspects of the inventive concepts will be described more fully hereinafter with reference to the accompanying drawings, in which some exemplary embodiments are shown. The present inventive concept may, however, be embodied in many different forms and should not be construed as limited to the exemplary embodiments set forth herein.

It will be understood that, although the terms first, second, etc. are be used herein to describe various elements, these elements should not be limited by these terms. These terms are used to distinguish one element from another, but not to imply a required sequence of elements. For example, a first element can be termed a second element, and, similarly, a second element can be termed a first element, without departing from the scope of the present invention. As used herein, the term “and/or” includes any and all combinations of one or more of the associated listed items.

It will be understood that when an element is referred to as being “on” or “connected” or “coupled” to another element, it can be directly on or connected or coupled to the other element or intervening elements can be present. In contrast, when an element is referred to as being “directly on” or “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.).

The terminology used herein is for the purpose of describing particular embodiments only and is not intended to be limiting of the invention. As used herein, the singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, steps, operations, elements, and/or components, but do not preclude the presence or addition of one or more other features, steps, operations, elements, components, and/or groups thereof.

Spatially relative terms, such as “beneath,” “below,” “lower,” “above,” “upper” and the like may be used to describe an element and/or feature's relationship to another element(s) and/or feature(s) as, for example, illustrated in the figures. It will be understood that the spatially relative terms are intended to encompass different orientations of the device in use and/or operation in addition to the orientation depicted in the figures. For example, if the device in the figures is turned over, elements described as “below” and/or “beneath” other elements or features would then be oriented “above” the other elements or features. The device may be otherwise oriented (e.g., rotated 90 degrees or at other orientations) and the spatially relative descriptors used herein interpreted accordingly.

In accordance with the inventive concepts, there is provided a removable mask airflow device, which can be removably coupled to a facemask. In various embodiments, the removable mask airflow device comprises a first portion constructed and arranged to be positioned at an exterior surface of a facemask, the first portion comprising an opening and at least one flow processing device disposed at the opening and a second portion constructed and arranged to be positioned at an interior surface of the facemask. The at least one flow processing device is configured to process fluid flow through the opening. The second portion is constructed and arranged to couple with the first portion with a portion of the facemask being between the first portion and the second portion. A flow processing device can take the form of a fan, a filter, and/or an UV emitter, as examples. Among various benefits, the removable mask airflow device in the example embodiments avoids foggy eyeglasses, controls moisture and wicks away sweat on the face, and helps ventilate for the user to enable more comfortable breathing, and can preferably be coupled to any facemask. Such facemasks can include, but are not limited to, N95, KN95, and other types of breathable facemasks.

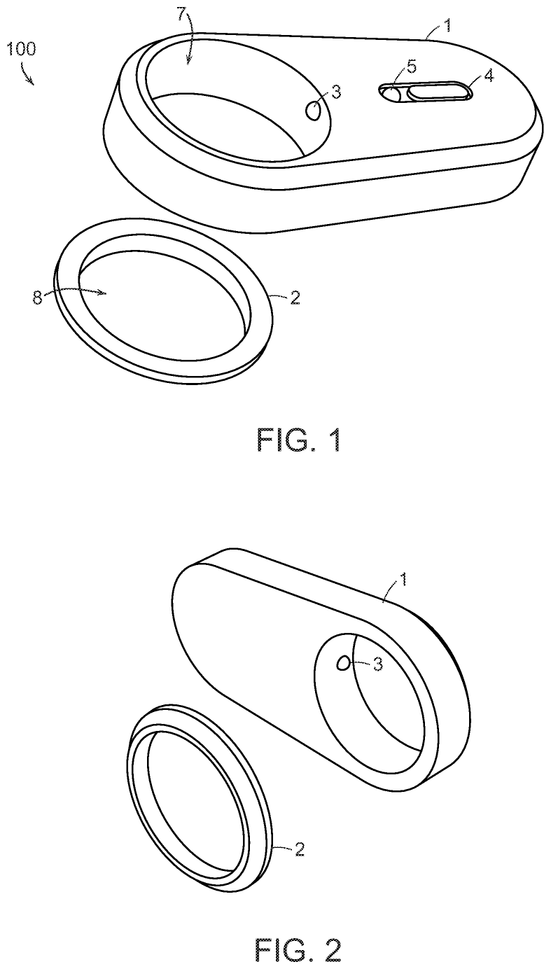

FIG. 1 is a front-perspective view of an embodiment of a removable mask airflow device 100, in accordance with aspects of inventive concepts. The removable mask airflow device is configured to removably attached to a facemask, such as a breathable facemask. In some embodiments, such as the one shown in FIG. 1, the removable mask airflow device 100 comprises a first portion 1 constructed and arranged to be positioned at an exterior surface of a facemask, the first portion 1 defining an opening 7. The opening 7 defines part of a flow path through the removable mask airflow device 100 and the facemask to which it is coupled.

In some embodiments, such as the one shown in FIG. 1, the removable mask airflow device 100 comprises a second portion 2 constructed and arranged to be positioned at an interior surface of the facemask. In some embodiments, such as the one shown in FIG. 1, the second portion 2 is constructed and arranged to removably couple with the first portion 1 with a portion of the facemask being fixed between the first portion 1 and the second portion 2. In this embodiment, the second portion 2 take the form of a ring, or is ring-shaped, defining an opening 8. In various embodiments, the opening 8 can be the same size, or substantially the same size, as the opening 7 of the first portion 1. The first and second portions 1, 2 are constructed and arranged such that, when coupled together, the openings 7, 8 align to form a flow path through the removable mask airflow device 100 and the portion of a mask disposed between the first and second portions 1, 2.

In some embodiments, such as the one shown in FIG. 1, the removable mask airflow device 100 comprises one or more emitters 3 at, extending from, and/or disposed within an inner surface of the opening 7. The one or more emitters 3 can be constructed and arranged to be directed towards at least a portion of the opening 7 to treat the flow passing therethrough. In some embodiments, the one or more emitters 3 emit radiation configured to degrade and/or destroy airborne living contaminants that pass through the opening 7, such as viruses, bacteria and the like. For example, in some embodiments, such as the one shown in FIG. 1, the emitter 3 is an ultraviolet (UV) emitter that emits UV radiation, which may degrade and/or destroy airborne viruses and/or bacteria. In alternative embodiments, the one or more emitters 3 can emit different forms of radiation configured to target different material or contaminants. The embodiment shown in FIG. 1, comprises one emitter 3, however alternative embodiments can comprise a plurality of emitters, e.g., distributed peripherally at or in the inner surface of the opening 7. In some embodiments, the plurality of emitters can include different types of emitters constructed and arranged to degrade and/or destroy different types of viruses and/or bacteria.

In some embodiments, the first portion 1 of the removable mask airflow device 100 comprises one or more batteries. The one or more batteries may provide energy to the different components of the first portion 1, including, but not limited to, the one or more emitters 3 and/or one or more fans 20 (see FIG. 10, 11). In some embodiments, the removable mask airflow device 100 comprises a 3.7 V lithium battery with a capacity of 3000 mAh, which can be rechargeable with about a four-hour charging time, as an example. In alternative embodiments, the battery can have different characteristics. Other types of rechargeable batteries could be used, including a solar battery. In some embodiments, the one or more battery could include a replaceable battery, such as a non-rechargeable battery, such as disk batteries.

In some embodiments, such as the one shown in FIG. 1, the removable air regulator device 100 comprises a USB Type-C charging port 6 (not shown in FIG. 1, see FIG. 6) configured to allow for charging the one or more batteries. In alternative embodiments, the removable mask airflow device 100 can comprise a different type of charging port. In alternative embodiments, the charging port 6 is located at a different location. In some embodiments, the charging port 6 can comprise a protective cover. In some embodiments, the protective cover comprises silicone.

In some embodiments, such as the one shown in FIG. 1, the removable mask airflow device 100 comprises a control button 4. In some embodiments, the control button 4 is configured to control the functional elements of the removable mask airflow device 100. As examples, the control button can be configured to turn ON and OFF a fan (see FIGS. 10 and 11) and/or emitters 3 of the apparatus. For example, via the control button 4, the user may turn ON/OFF one or more emitters 3, turn ON/OFF one or more fans, and/or control the speed of a variable speed fan. In some embodiments, using the control button 4 some emitters 3 may be turned ON, while others are turned OFF. In some embodiments, the control button 4 may be used to configure the one or more emitters 3 to automatically turn ON and OFF at a specific predefined frequency or duration. In the embodiment shown in FIG. 1, the removable mask airflow device 100 comprises one control button 4. In alternative embodiments, the removable mask airflow device 100 can comprise a different number of control buttons 4.

In some embodiments, such as the one shown in FIG. 1, the removable mask airflow device 100 comprises one or more status indicators 5. The one or more status indicators are each configured to display a signal corresponding to different mode configurations. For example, the status indicator may take the form of or include one or more light-emitting diodes (LEDs). An indicator may light one color when the emitter 3 is ON, another color when the emitter 3 is OFF, and a third color when the battery is fully charged, etc. Different status indicator states may relate to the operation of the fan, e.g., relate to or indicate fan ON/OFF and/or fan speed.

In some embodiments, such as the one shown in FIG. 1, the first portion 1 of the removable air regulator device 100 comprises a printed circuit board (PCB) (not shown). In some embodiments, the PCB includes various functional circuit components that allow for electrical coordination of power and control functions of the removable mask airflow device device.

In some embodiments, such as the one shown in FIG. 1, the first portion 1 comprises an elongated shape. In alternative embodiments, the first portion 1 comprises a different shape including, but not limited to, a circle, a square, a rectangle, a triangle, an oval, a hexagon, an octagon, or any such suitable polygon, or combinations thereof.

In some embodiments, such as the one shown in FIG. 1, the opening 7 comprises a circular shape. In alternative embodiments, the opening 7 can comprise a different shape including, but not limited to, a square, a rectangle, a triangle, an oval, a hexagon, an octagon, or any such suitable polygon or combinations thereof.

In some embodiments, such as the one shown in FIG. 1, the second portion 2 is constructed and arranged to removably couple with the first portion 1 with a portion of the facemask being fixed between the first portion 1 and the second portion 2. In some embodiments, such as the one shown in FIG. 1, the second portion 2 comprises a circular shape. In alternative embodiments, the second portion 2 can comprise a different shape including, but not limited to a square, a rectangle, a triangle, an oval, a hexagon, an octagon, or any such suitable polygon or combinations thereof.

In some embodiments, such as the one shown in FIG. 1, the second portion 2 comprises the opening 8 constructed and arranged to align with the opening 7 of the first portion 1 when the first and second portions are coupled together. In this embodiment, the second portion 2 takes the form of a ring having the center opening 8 that is the same or about the same size as the opening 7 of the first portion. In some embodiments, such as the one shown in FIG. 1, the opening 8 comprises a circular shape. In alternative embodiments, the opening 8 can comprise a different shape including, but not limited to a square, a rectangle, a triangle, an oval, a hexagon, an octagon, or any such suitable polygon or combinations thereof.

In some embodiments, such as the one shown in FIG. 1, the he first portion 1 and the second portion 2 magnetically couple together, to form a removable coupling. In some embodiments, the second portion 2 comprises one or more magnets over-molded by silicone. These magnets mate with corresponding magnets on the first portion 1. In alternative embodiments, the second portion 2 is formed and/or comprises different materials. In alternative embodiments, the second portion 2 is coupled to the first portion 1 by another mechanism, including, but not limited to, hooks, buttons, compression fit, snap fit, a sliding mechanism, or any such suitable attachment mechanism or combinations thereof that create a removable coupling between the first and second portions 1, 2.

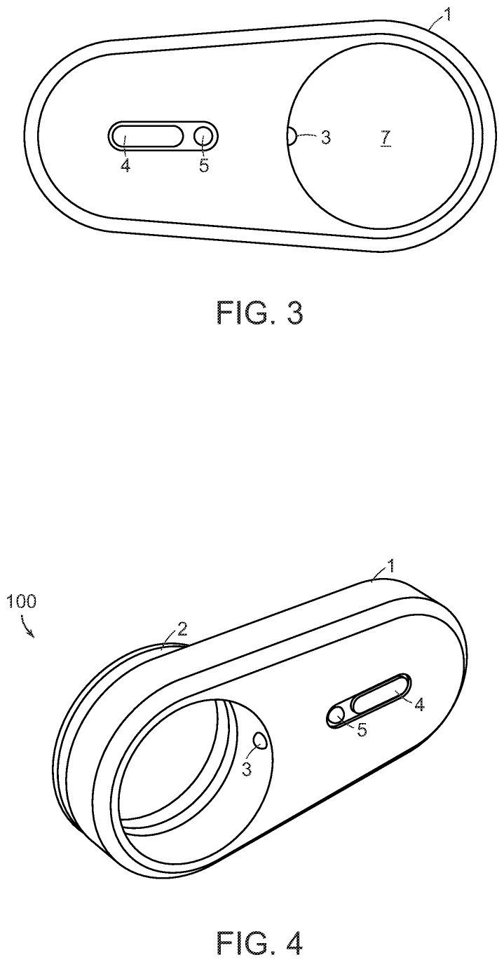

FIG. 2 is a rear-perspective view of the embodiment of FIG. 1, in accordance with aspects of inventive concepts. This is a dissembled view, where the first 1 and second portions 2 are not coupled together.

FIG. 3 is a front view of the embodiment of FIG. 1, in accordance with aspects of inventive concepts. This view shows the control button(s) 4 and status indicator(s) 5, which remain accessible external to the mask when worn.

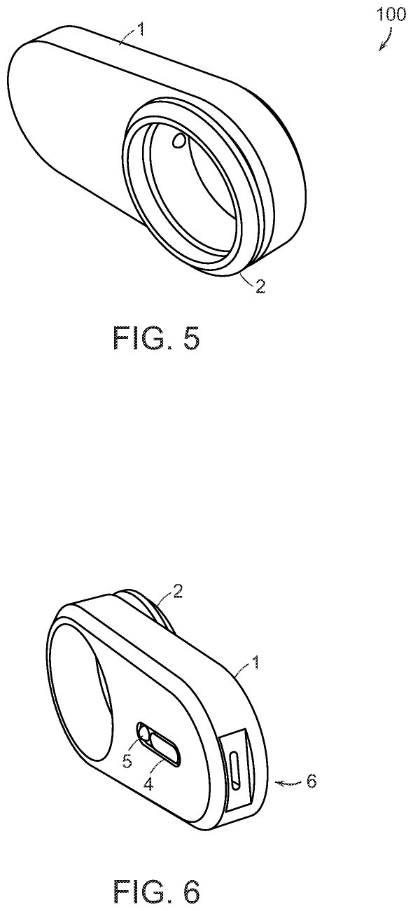

FIG. 4 is a front-perspective view of the embodiment of FIG. 1, in accordance with aspects of inventive concepts. In the embodiment shown in FIG. 4, the opening 8 of the second portion 2 is aligned with the opening 7 of the first portion 1 when the two portions are coupled together (as shown).

FIG. 5 is a rear-perspective view of the embodiment of FIG. 1, in accordance with aspects of inventive concepts. As in FIG. 4, the first and second portions are coupled together. A mask is omitted to aid in the understanding of the engagement of the first and second portions.

FIG. 6 is a front-perspective view of the embodiment of FIG. 1, in accordance with aspects of inventive concepts. In this view the USB Type-C charging port 6 is visible at a bottom end of the removable mask airflow device 100. The charging portion 6 is configured to allow for charging the one or more batteries. In alternative embodiments, the removable air regulator device 100 can comprise a different type of charging port. In alternative embodiments, the charging port 6 is located at a different location. Preferably, in at least some embodiments, the charging port remains accessible while the device is in use. In still other embodiments, the removable mask airflow device can be configured for wirelessly charging.

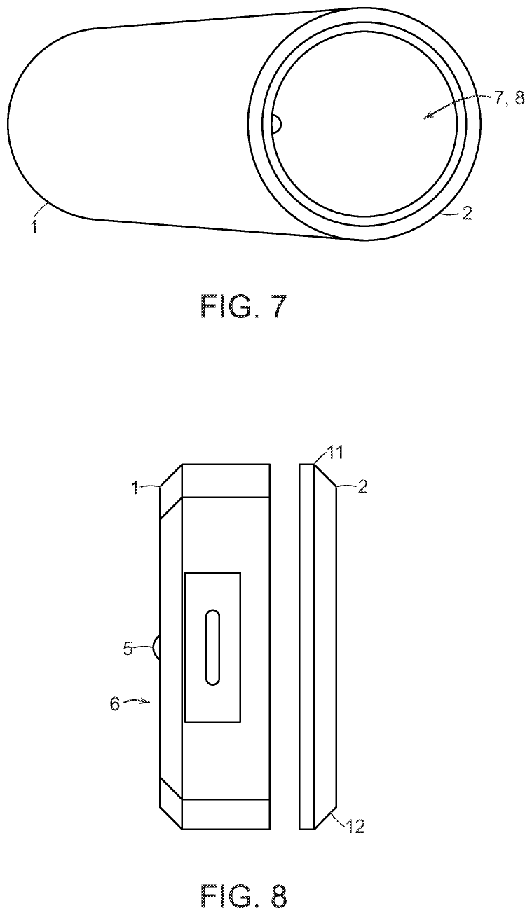

FIG. 7 is a rear view of the embodiment of FIG. 1, in accordance with aspects of inventive concepts, showing the first 1 and second 2 portions engaged.

FIG. 8 is a bottom view of the embodiment of FIG. 1, in accordance with aspects of inventive concepts. In the view shown in FIG. 8, the first portion 1 and the second portion 2 are spaced apart and disengaged. In some embodiments, during use, a facemask would fit in the space between the first portion 1 and the second portion 2. In the embodiment shown in FIG. 8, the second portion 2 comprises a beveled edge having flat region 11 and an angled region 12, which forms a taper. In alternative embodiments, the second portion 2 can be configured differently, without a straight edge and/or a beveled edge. In some embodiments, the second portion can have curved edges or some other form of edge.

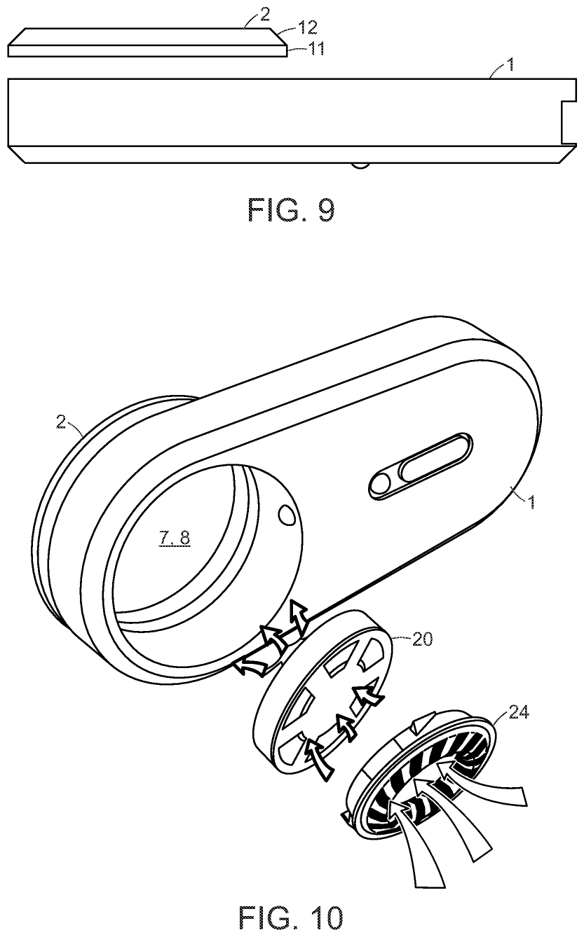

FIG. 9 is a side view of the embodiment of FIG. 1, in accordance with aspects of inventive concepts. In the view shown in FIG. 9, the first portion 1 and the second portion 2 are spaced apart, disengaged. In some embodiments, during use, a facemask would fit in the space between the first portion 1 and the second portion 2. In the embodiment shown in FIG. 9, the flat portion 11 and beveled edge 12 of the second portion 2 are apparent. In alternative embodiments, the second portion 2 can be configured differently, as previously discussed.

FIG. 10 is a front-perspective, partially-exploded view of an embodiment of the removable mask airflow device 100 of FIG. 1, in accordance with aspects of inventive concepts. In this embodiment, the removable mask airflow device 100 comprises a fan 20. In some embodiments, the fan 20 can be optional and the removable mask airflow device 100 can still include at least one emitter 3. In other embodiments, the removable mask airflow device 100 can include the fan 20, but no emitters 3. In still other embodiments, the removable mask airflow device 100 can include the fan 20 and at least one emitter 3.

In some embodiments, such as the one shown in FIG. 10, the removable mask airflow device 100 comprises one or more fan modules 20 and one or more corresponding fan covers 24. In some embodiments, such as the one shown in FIG. 10, the fan module 20 is an axial fan module. The fan module 20 and the fan cover 24 may be positioned and/or fit within the opening 7 of the first portion 1. In alternative embodiments, the device 100 comprises multiple openings and multiple fan modules, each corresponding to a respective opening in the first portion. In some embodiments, one or more fan modules 20 can be easily removed from the first portion 1, as can one or more fan covers 24.

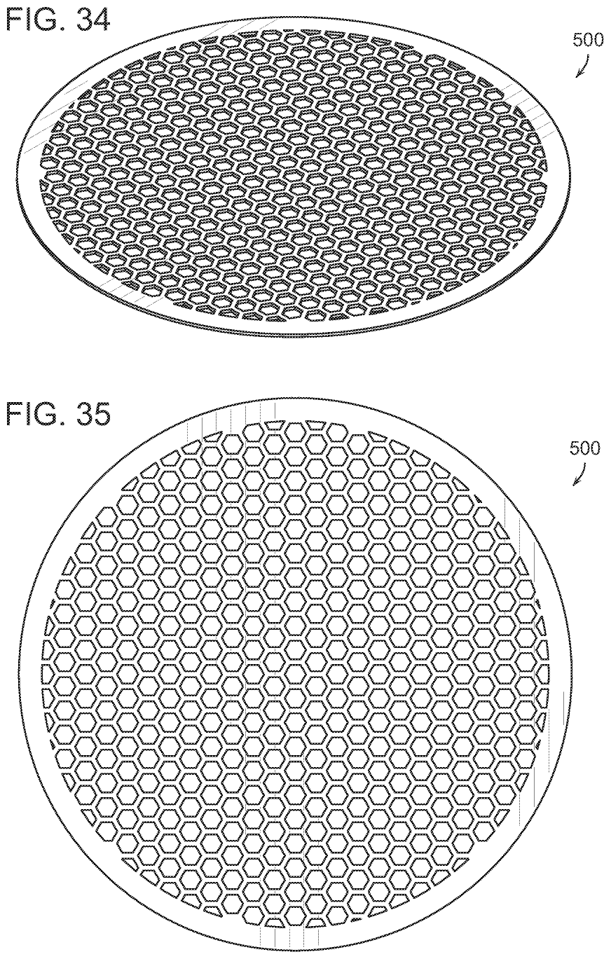

In some embodiments, the one or more fan modules 20 and/or the respective covers 24 can be removable, e.g., threaded or press fit onto the first portion 1. In some embodiments, an air filter (see FIGS. 34-49) can be disposed between the one or more fan modules 20 and the respective covers 24. Such an air filter could be reusable and washable or could be single-use disposable.

In some embodiments, the one or more fan modules 20 comprises a cordless, battery operated motor. In some embodiments, one or more of the fan modules 20 can be constructed and arranged to operate at about 10,000 rpm or faster. But in other embodiments, the one or more fan modules 20 can be constructed and arranged to operate at a different speed. In some embodiments, the one or more fan modules 20 can include a variable-speed fan that operates at different speeds, and such variable speeds can be user controlled, e.g., through control button 4. In some embodiments, a user-selected speed setting is stored can be a memory on the PCB such that the user may turn off the fan at a speed setting and when the fan is turned on again it will be turned on at the same speed setting. In some embodiments, the one or more fan modules 20 can process a fan air volume of up to or about 1.9 cubic feet per minute. In alternative embodiments, the one or more fan modules 20 can process a different and/or variable fan air volume.

In some embodiments, the removable mask airflow device 100 can comprise an ionizer constructed and arranged to ionize the air before it passes through each of the one or more fan modules 20. In some embodiments, the ionizer can be removable.

In some embodiments, the removable mask airflow device 100 can comprise one or more filters constructed and arranged to filter the air before it passes through the one or more fan modules 20 (see, e.g., filter examples of FIGS. 34-49). In some embodiments, one or more of the filters can be removable and, optionally, washable and/or replaceable. In some embodiments, one or more of the filters can be an anti-bacterial filter. In some embodiments that include a filter, the device 100 does not include an emitter 3. But in some embodiments that include a filter, the device 100 also includes one or more emitters 3, as previously described.

In some embodiments, the one or more fan modules are powered by the batteries within the first portion 1, and charged through the USB Type-C charging port 6 or wirelessly. In some embodiments, one or more of the one or more fan modules 20 can operate while the one or more batteries are charging. In some embodiments, one or more of the one or more fan modules 20 cannot operate while the one or more batteries are charging.

In some embodiments, the one or more fan modules 20 can operate at different speeds through operation of one or more control mechanisms, such as the control button 4 shown in FIG. 10. For example, the one or more fans 20 could selectively operate at a low speed, a medium speed, or a high speed. In some embodiments, the fan control mechanism could take the form of a fan control button, e.g., control button 4, and a user could cycle through different speed settings and/or direction settings by pushing the fan control button multiple times. For example, pressing the fan control button once could turn on the fan 20 at the low speed, pressing the button a second time could cause the fan 20 to operate at the medium speed, and pressing the button a third time could cause the fan to operate at the high speed. Pressing the button a fourth time, or holding the button down, could turn off the fan 20.

In other embodiments, the different fan speed settings could be assigned independent fan control buttons: one for the low speed, one for the medium speed, one for the high speed, etc. In some embodiments, the fan control mechanism could take the form of a potentiometer that enables the user to adjust fan speed over a continuum. In some embodiments, the fan control button is the same as the control button 4. In alternative embodiments, the fan control button is independent of the control button associated with the emitter 3. In some embodiments, a user-selected speed setting is stored in a memory such that the user may turn off the fan at a selected speed setting and when the fan is turned on again it will still be at the selected speed setting.

In some embodiments, such as the one shown in FIG. 10, the one or more fan modules 20 is configured to blow air towards the user and pull air from the environment external to the mask. Such configurations would regulate air that the user inhales. In alternative embodiments, one or more of the one or more fan modules 20 is configured to blow exhaled breath from internal to the mask to the external environment, pulling the exhaled breath away from the user. Such configurations would regulate the breath exhaled by the user. In some embodiments, the fan control mechanism allows the user to easily switch between these two fan configurations, e.g., by switching fan rotation direction. The same control button, or a different user-manipulatable mechanism, could be used to change fan direction. In some embodiments, the device 100 could include at least one fan blowing air into the mask and at least one other fan blowing exhaled breath external to the mask. In some embodiments, each fan module can include an emitter 3 and/or filter.

FIG. 11 is a front-perspective view of the device of FIG. 10, in accordance with aspects of inventive concepts. In some embodiments, such as the embodiments shown in FIG. 11, the second portion 2 is in the form of a ring that comprises three magnets 9 at least partially over-molded by silicone. These magnets mate with corresponding magnets and/or material on the first portion 1 to facilitate magnetic coupling between the first 1 and second portions 2. In alternative embodiments, the second portion 2 comprises a different number of magnets. In alternative embodiments, the second portion 2 is formed of and/or comprises different materials, and the first portion comprises one or more magnets 9. In alternative embodiments, the first 1 and second portions 2 are coupled via a different attachment mechanism, as discussed above.

In the view shown in FIG. 11, the fan module 20 and cover 24 are installed in the first portion 1 of the device 100. In alternative embodiments, the fan module 20 and the cover 24 an be installed in the second portion 2 of the device 100.

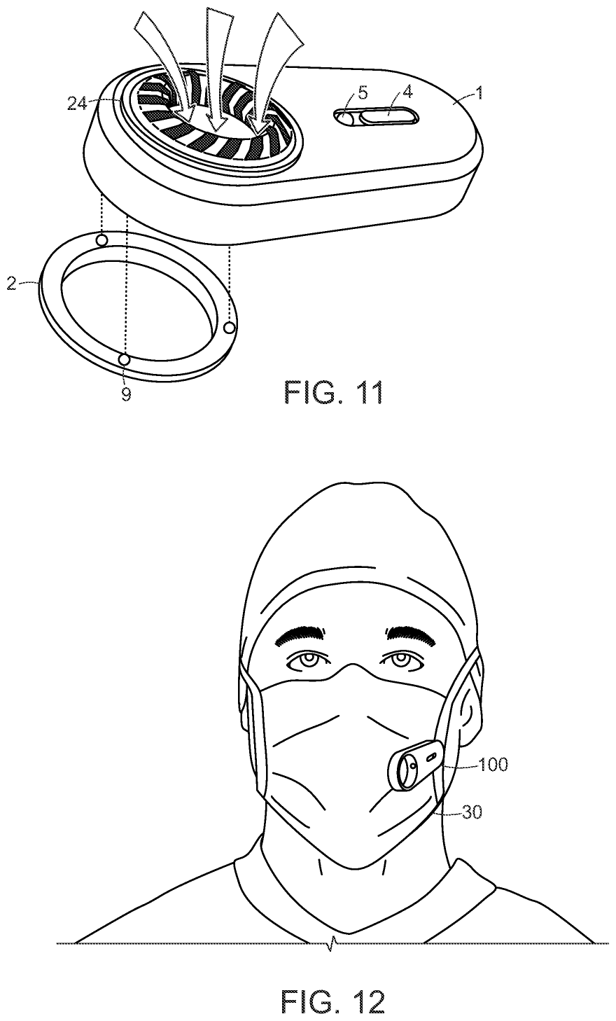

FIG. 12 shows a user wearing an embodiment of the removable mask airflow device 100 of FIG. 1, in accordance with aspects of inventive concepts. In this embodiment, the device 100 is coupled to a facemask 30, with the first portion 1 external to the mask and the second portion 2 internal to the mask 30. The first portion 1 is coupled to the mask 30 using the second portion 2 with a magnetic coupling therebetween, as previously described. In this embodiment, some of the air (and other gases, aerosols, etc.) that pass through the opening 7 of the first portion 1, may be exposed to the ultraviolet radiation previously described from the emitter(s) 3. Such radiation may degrade and/or destroy material, e.g., viruses and bacteria, that is incoming or outgoing relative to the user and the mask. In various embodiments, the air (and other gases, aerosols, etc.) that pass through the opening 7 of the first portion 1 may, additionally or alternatively, be exposed to at least one filter, which may have antiviral and/or antibacterial properties and/or coatings. In the view shown, the removable air regulator device 100 is at the left side of the user's face. The device 100 would also work at different locations on the mask 30.

In FIG. 12, the device 100 is shown without a fan module 20 and fan cover 24, but could include the foregoing in other embodiments.

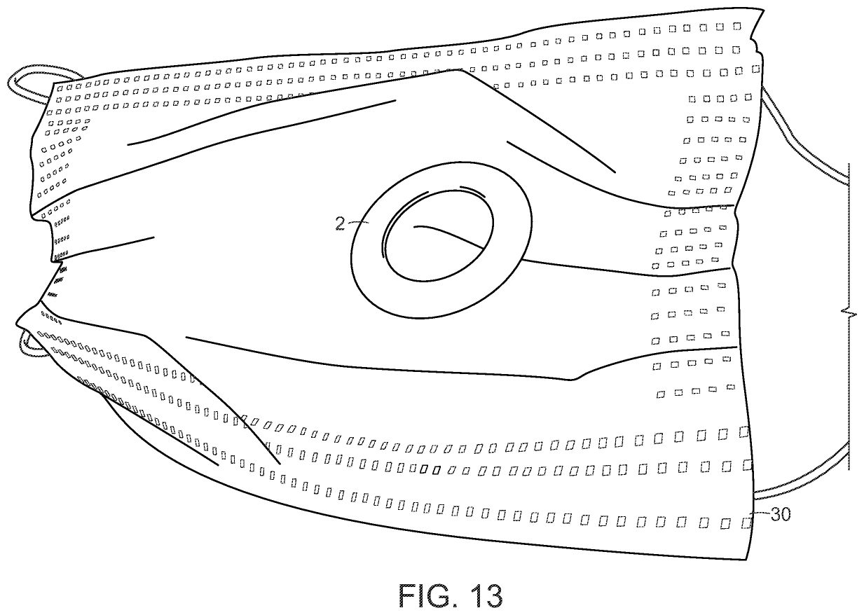

FIG. 13 is a rear-perspective view of an embodiment of the removable mask airflow device 100 coupled to a mask 30, in accordance with aspects of inventive concepts. In this view, the second portion 2 is shown at an interior surface of the mask 30.

In some embodiments, the removable mask airflow device 100 can comprise a thermometer. In some embodiments, the thermometer can be configured to communicate with a network, e.g., via Bluetooth technology.

In some embodiments, the first portion 1 can comprise other forms of attachment mechanism for coupling to the mask 30. In some embodiments, the attachment mechanism can include a clip, a compression fit mechanism, a snap fit mechanism, a sliding mechanism, or any such suitable attachment mechanism or combinations thereof.

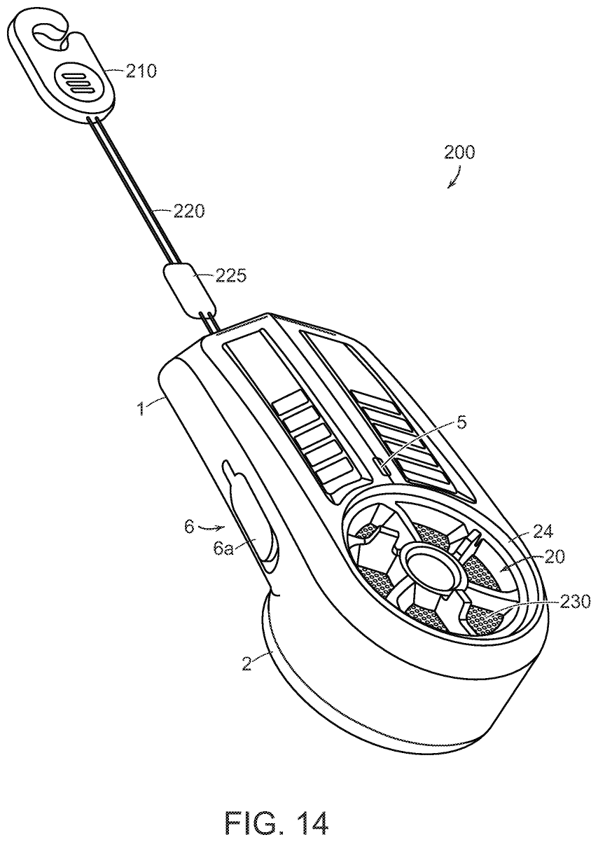

FIG. 14 is a perspective view of an alternative embodiment of a removable mask airflow device 200, in accordance with aspects of inventive concepts. Similar to the embodiments previously described, in this embodiment the device 200 comprises a first portion 1 constructed and arranged to couple with a second portion 2, with a facemask between the first portion 1 and the second portion 2. Similar to the previously described embodiments, one or more fan modules can be mounted within the first portion 1. In the view shown in FIG. 14, the device is in assembled form, without a mask disposed between the first and second portions.

The embodiment shown in FIG. 14 includes a connector 210 constructed and arranged to securely couple the air regulator device to another item. In the embodiment shown in FIG. 14, an elongated member 220 couples the connector to the first portion 1. In some embodiments, the elongated member is a rope, chain, wire, cord, or any such suitable member that can couple the connector 210 and the first portion 1. In some embodiments, the elongated member 220 is flexible. In alternative embodiments, the elongated member 220 is rigid. In some embodiments the distance between the connector 210 and the first portion 1 is adjustable. In the embodiment shown in FIG. 14, the device 200 comprises an adjustor 225 configured to adjust a length of the elongated member 220, such that the distance between the connector 210 and the first portion 1 is adjustable. In some embodiments, the connector 210 can be directly connected to the first portion 1. In some embodiments, the connector 210 can include a clip, button, hook, hook and loop, a compression fit mechanism, a snap fit mechanism, a sliding mechanism, or any such suitable attachment mechanism or combinations thereof.

In one example embodiment, the connector 210 can be attached on one of the threads of the face mask to further secure the removable airflow device 200 on the facemask. In other example embodiments, the connector 210 can be attached on any part of the facemask. In other example embodiments, the connector 210 can be attached on any wearable apparatus of the user. For example, the connector 210 can be attached on the glasses of the user. When the removable airflow device 200 is not in use, the user can detach the removable airflow device 200 from the facemask and use the connector 210 to attach the removable airflow device 200 on the glasses of the user. The connector 210 provides convenience to the user to use the removable airflow device 200 and provide further securement when the removable airflow device 200 is displaced and detached from the facemask.

In the embodiment shown in FIG. 14, the charging port 6 is located at a side surface of the first portion 1. In some embodiments, such as the one shown in FIG. 14, the device 200 comprises a cover 6a for the charging port 6. In alternative embodiments, the air regulator device 200 does not comprise a cover for the charging port 6. In other embodiments, the device 200 can be wirelessly charged and the charging port 6 and cover 6a can be omitted.

In the embodiment of FIG. 14, a filter 230 is visible through the fan cover 24 and a fan module is disposed within the first portion 1, beneath the filter 230.

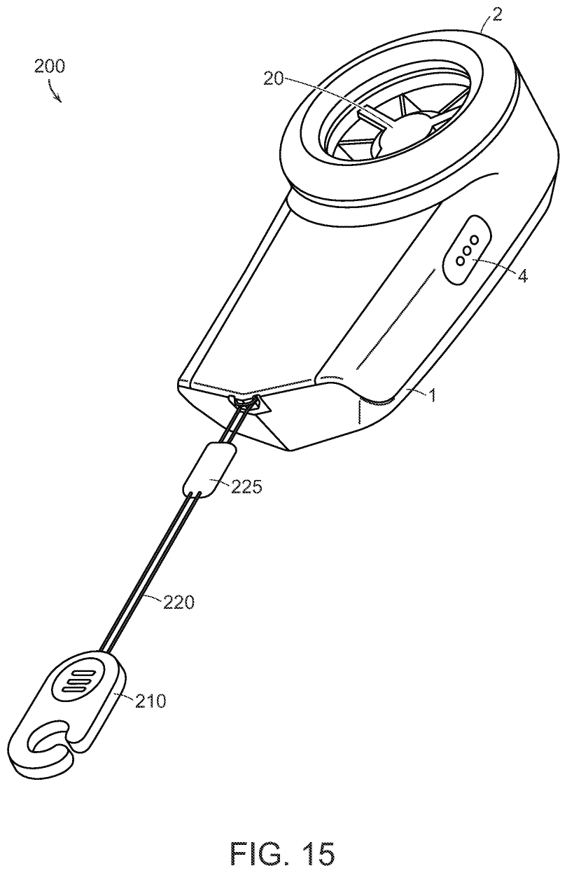

FIG. 15 is a perspective view of the embodiment of FIG. 14, in accordance with aspects of inventive concepts. In this view, the device 200 is in assembled form, without a mask disposed between the first and second portions. The fan module 20 is visible, as is the second portion 2 magnetically coupled to the first portion 1. A control button 4 is also visible.

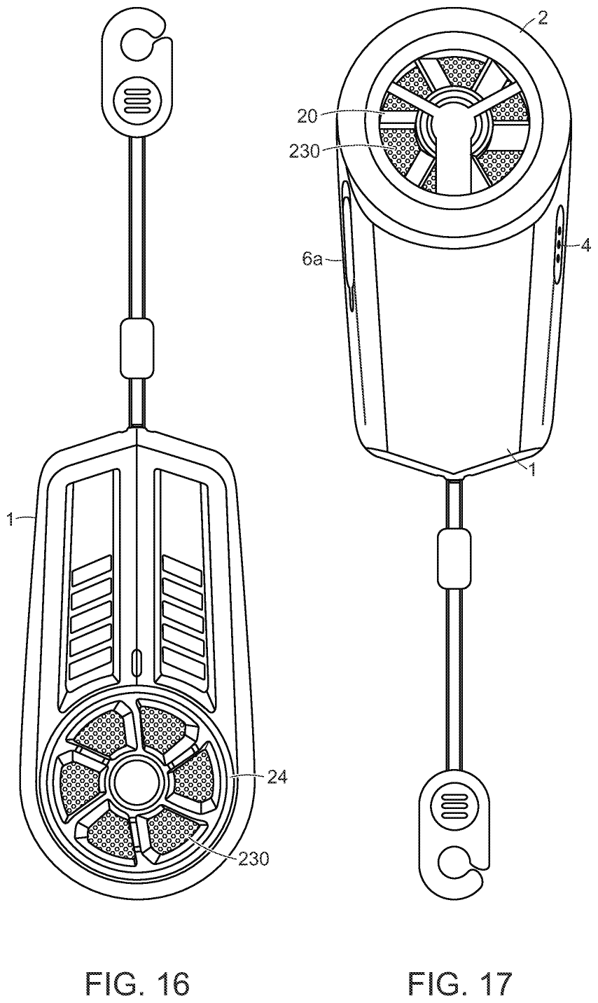

FIG. 16 is a front view of the embodiment of FIG. 14, in accordance with aspects of inventive concepts. In this view, as in FIG. 14, a status indicator 5 is visible.

FIG. 17 is a rear view of the embodiment of FIG. 14, in accordance with aspects of inventive concepts. The fan module 20 is visible, as is the filter 230. The second portion 2 is also visible.

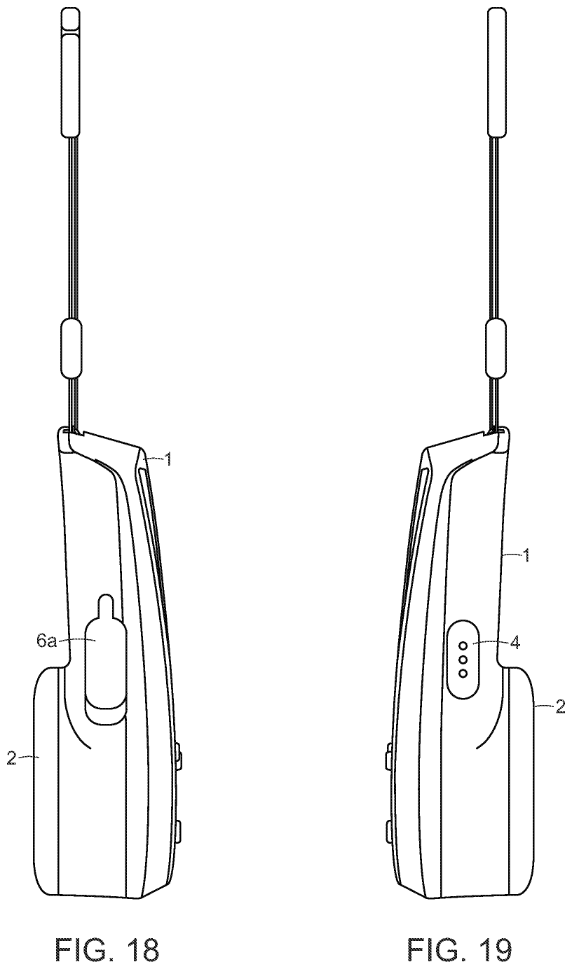

FIG. 18 is a first side view of the embodiment of FIG. 14, in accordance with aspects of inventive concepts. In this view, the device is in assembled form, without a mask disposed between the first and second portions. The charge port cover 6a is visible.

FIG. 19 is a second view of the embodiment of FIG. 14, in accordance with aspects of inventive concepts. In this view, the device is in assembled form, without a mask disposed between the first and second portions. In this view, the control button 4 is visible.

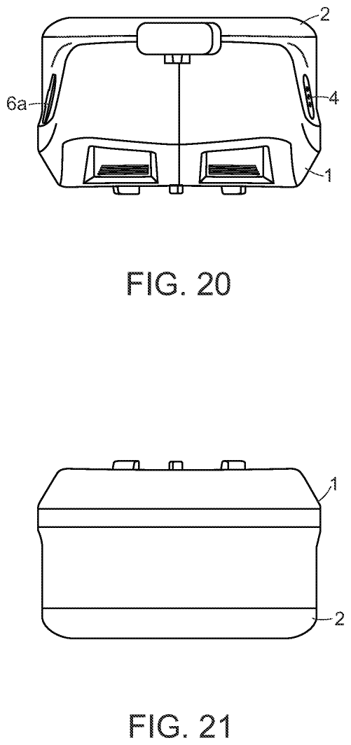

FIG. 20 is a bottom view of the embodiment of FIG. 14, in accordance with aspects of inventive concepts.

FIG. 21 is a top view of the embodiment of FIG. 14, in accordance with aspects of inventive concepts. In this view, the device is in assembled form, without a mask disposed between the first and second portions.

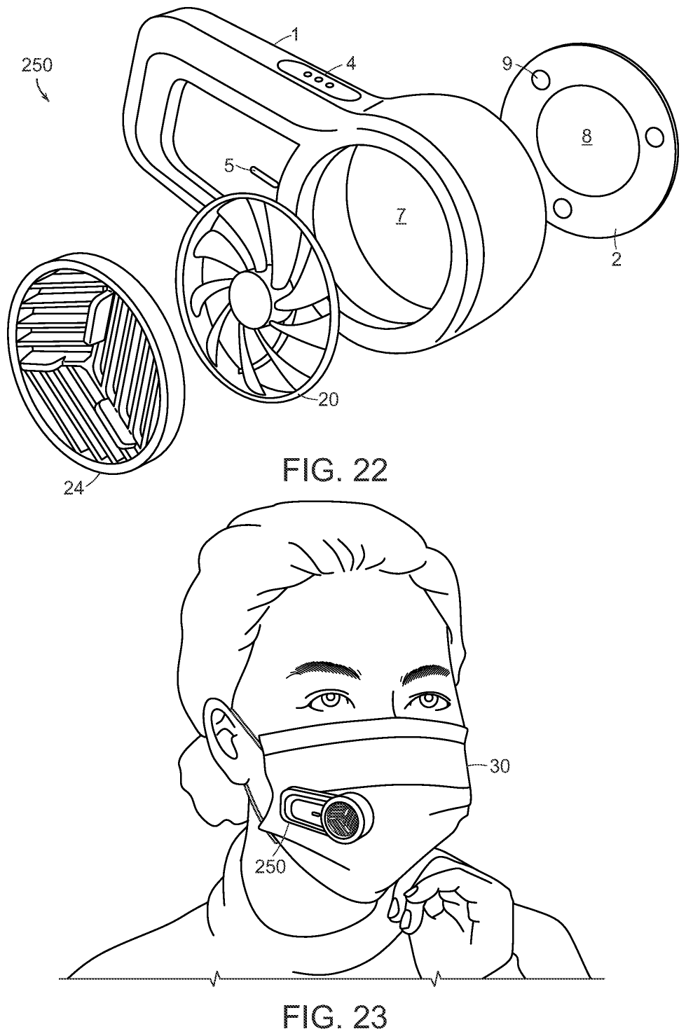

FIG. 22 is a front-perspective, partially-exploded view of another embodiment of a removable air regulator device 250, similar to the embodiment of FIG. 14, in accordance with aspects of inventive concepts. In some embodiments, such as the one shown in FIG. 22, the device 250 comprises one control button 4. In alternative embodiments, the device 250 comprises a different number of control buttons. In this embodiment, the control button 4 can control the speed (low or high) and power of the fan 20. In this embodiment, the opening 7 of the first portion 1 is aligned with the opening 8 of the second portion 2, such that air can flow through both openings 7, 8. In the embodiment shown in FIG. 22, the second portion 2 comprises three magnets 9 which couple to the first portion 1 to removable secure the device 250 on a mask 30.

In some embodiments, such as those shown in FIGS. 14 and 22, the device 200, 250 has a mass of around 40 g within a range of +/−20% inclusive. In alternative embodiments, the device 200 can have a different weight.

In some embodiments, such as those shown in FIGS. 14 and 22, the device 200, 250 is around 80 mm long by 40 mm by 25 mm, with each dimension being within a range of +/−20% inclusive. In alternative embodiments, the device 200 can have different dimensions.

In some embodiments, such as those shown in FIGS. 14 and 22, the device 200, 250 comprises a 550 mAh battery with the total charge being within a range of +/−20% inclusive. In alternative embodiments, the device 200 can comprise a battery with different characteristics.

In some embodiments, such as those shown in FIGS. 14 and 22, the device 200, 250 comprises a 30 mm axial fan 20 with the fan dimensions being within a range of +/−20% inclusive. In alternative embodiments, the device 200 can comprise a fan with different characteristics.

In some embodiments, such as those shown in FIGS. 14 and 22, the device 200, 250 comprises a USB Type-C charging port. In alternative embodiments, the device 200 comprises a charging port with different characteristics and/or is wirelessly chargeable.

In some embodiments, such as those shown in FIGS. 14 and 22, the first portion 1 can comprise or be formed from polypropylene (PP). In alternative embodiments, the first portion 1 can comprise or be formed from high-density polyethylene (HDPE), low-density polyethylene (LDPE), acrylonitrile butadiene styrene (ABS), polycarbonate (PC), polyethylene terephthalate (PET), polyamides (PA), polycarbonate/acrylonitrile butadiene styrene (PC/ABS), polystyrene (PS), polyurethane (PU), or combinations of two or more thereof. In alternative embodiments, the first portion 1 can additionally or alternatively comprise one or more different materials, so long as such materials maintain structural integrity.

In some embodiments, such as those shown in FIGS. 14 and 22, the second portion 2 comprises silicone over-molded with magnet pieces 9. In alternative embodiments, the second portion 2 can be formed of and/or comprises different materials.

In some embodiments, such as those shown in FIGS. 14 and 22, the filter 230 can comprise a stainless-steel mesh with a nano-silver coating. In alternative embodiments, the filter 230 can be formed of and/or comprise different materials.

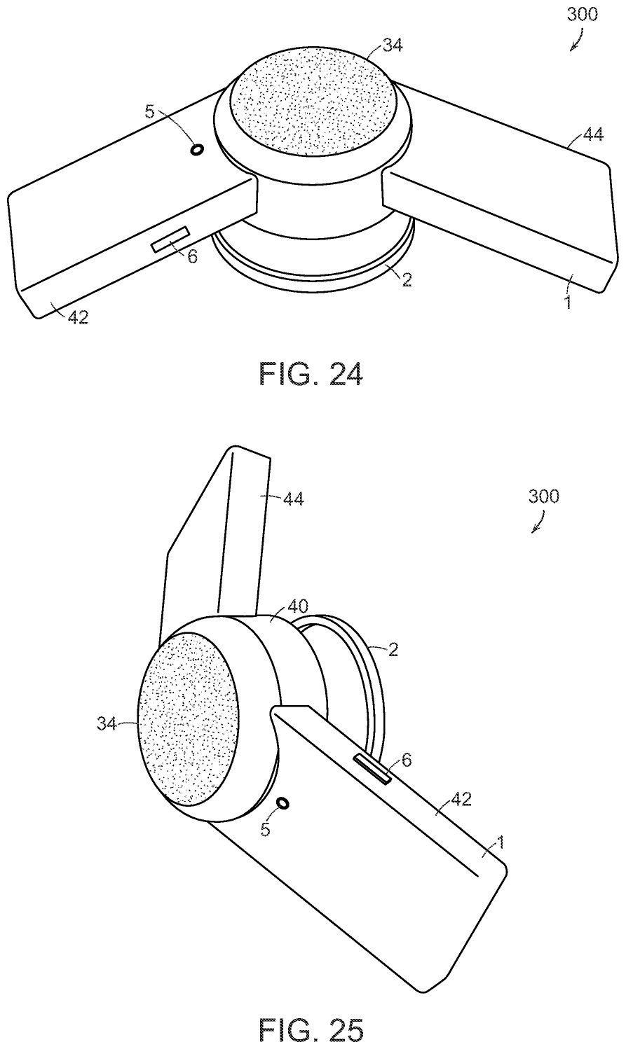

FIG. 23 shows a user wearing a mask 30 and the removable mask airflow device 250 of FIG. 22, in accordance with aspects of inventive concepts. In this embodiment, the device 250 is coupled to a facemask 30, using a second portion 2 interior to the mask, as previously described. In this embodiment, some of the air (and/or other gases, aerosols, etc.) that pass through the opening 7 of the first portion 1, may be exposed to the ultraviolet radiation and/or nano-silver coating previously described. Such exposure may degrade and/or destroy material that is incoming or outgoing relative to the user. In the view shown, the removable air regulator device 250 is at the right side of the user's face. The device 250 would also work at different locations on the mask 30.

In some embodiments, the removable mask airflow device 200, 250 can comprise a thermometer. In some embodiments, the thermometer can be configured to communicate with a network, e.g., using Bluetooth technology.

In some embodiments, the first portion 1 comprises an attachment mechanism for coupling to the mask. In some embodiments, the attachment mechanism can include a clip, button, hook, hook and loop, a compression fit mechanism, a snap fit mechanism, a sliding mechanism, or any such suitable attachment mechanism or combinations thereof.

Aside from the differences mentioned, the removable mask airflow device 200, 250 shown in FIGS. 14 and 22 may include different characteristics discussed in connection with other embodiments discussed herein.

FIG. 24 is a front-perspective view of an alternative embodiment of a removable mask airflow device 300, in accordance with aspects of inventive concepts. Similar to the embodiments previously described, in this embodiment the device 300 comprises a first portion 1 constructed and arranged to couple with a second portion 2 with a facemask between the first portion 1 and the second portion 2. Similar to the previously described embodiments, one or more fan modules (not shown) can mounted within the first portion 1, such as in one or more openings thereof.

In some embodiments, such as the one shown in FIG. 24, the first portion 1 comprises a central region 40. In some embodiments, such as the one shown in FIG. 24, the central region 40 comprises one or more fan modules 20. The central region 40 also includes an opening 7 (not visible) that defines a flow path through the device 300. The fan module 20 may be disposed within the opening 7 and flow path.

In some embodiments, such as the one shown in FIG. 24, the second portion 2, which is shown spaced apart from the first portion 1, is constructed and arranged to be removably and magnetically coupled to the first portion 1. In some embodiments, such as the one shown in FIG. 24, when the second portion 2 is coupled to the first portion 1, the opening of the first portion 1 is aligned with the opening 8 of the second portion 2, to form a flow path therethrough.

In some embodiments, such as the one shown in FIG. 24, the second portion 2 is in the form of a magnet ring. In some embodiments, such as the one shown in FIG. 24, the second portion 2 comprises silicone molded with one or more magnets 9, as discussed above. In alternative embodiments, the second portion 2 can be formed of and/or comprises different materials.

In some embodiments, such as the one shown in FIG. 24, the first portion 1 of the removable mask airflow device 200 comprises one or more legs coupled to and/or extending from the central region 40. In the embodiment shown in FIG. 24, the first portion 1 comprises two legs 42, 44. In alternative embodiments, the first portion 1 comprises a different number of legs.

In some embodiments, the one or more legs 42, 44 are inclined relative to the bottom of the central region 40 and toward a mask. In some embodiments, the one or more legs are inclined relative to each other. In the embodiment shown, the legs 42, 44 at least partially conform to a face, and at least partially encompass the second portion 2.

In some embodiments, such as the one shown in FIG. 24, the first portion 1 comprises a cover 34 at one (front) side of the fan module 20. In some embodiments, such as the one shown in FIG. 24, the cover 34 is removable. In some embodiments, such as the one shown in FIG. 24, the cover 34 comprises a mesh that can serve as a filer to block contaminants. The cover 34 shown in FIG. 24 can be configured so that it can be easily washed or it can be disposable and replaceable.

In the embodiment shown in FIG. 24, a PCB can be stored within the first leg 42. In alternative embodiments, the PCB may be located in the second leg, or there can be a PCB in each leg.

In the embodiment shown in FIG. 24, the battery can be stored within the second leg 44, e.g., when the PCB is in the first leg 42. In alternative embodiments, the battery may be first leg 42 and the PCB can be in the second leg 44. In other embodiments, the a leg 42, 44 can include a PCB and a battery.

In the embodiment shown in FIG. 24, the charging port is located in the first leg 42. In alternative embodiments, the charging port may be at a different leg. In some embodiments, wireless charging capability can be built into the first portion 1, e.g., in the first or second leg 42, 44.

In the embodiment shown in FIG. 24, the status indicator 5 is at the first leg 42. In alternative embodiments the status indicator may be in the second leg 44. In still other embodiments, there may be at least one status indicator in each leg.

There are some additional differences between the embodiment 300 shown in FIG. 24 and the embodiments previously described. The embodiment shown in FIG. 24 is constructed and arranged to be positioned on the facemask near the user's mouth, increasing the observability of the device 300. The embodiment shown in FIG. 24 also has a larger footprint than the embodiments previously described. If the one or more fan modules 20 are configured to blow air towards the user, a larger device 300 that is closer to the user's mouth may result in filtered air being more efficiently delivered to the user's mouth. If the one or more fan modules 20 are configured to blow air away from the user, a larger device 300 that is closer to the user's mouth may result in exhaled breath being more efficiently directed to the opening of the first portion 1.

The weight of the embodiment shown in FIG. 24 is more balanced than the weight of the previously described embodiments, largely due to the more symmetric structure of the first portion 1. In some embodiments, such as the one shown in FIG. 24, the sides of the device 300 can twist, bend, hinge, or otherwise be user-manipulatable and/or rotatable for user's comfortability. Aside from the differences mentioned, the embodiment shown in FIG. 24 may include the same or different characteristics previously discussed in connection with the embodiments shown in previous figures.

FIG. 25 is a front-perspective view of the embodiment of FIG. 24, in accordance with aspects of inventive concepts, with the second portion detached from the first portion.

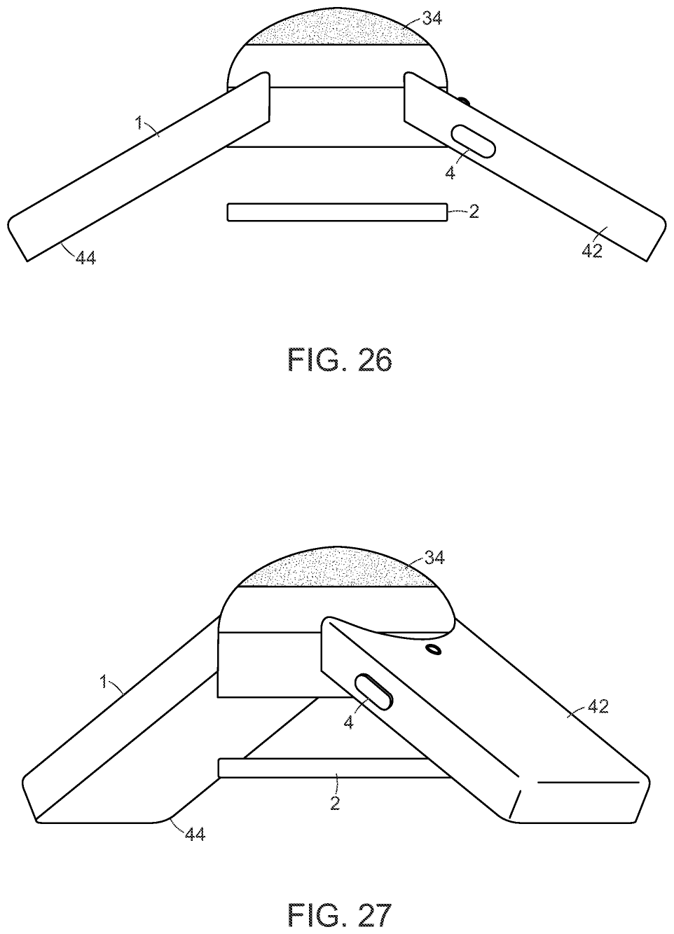

FIG. 26 is a top view of the embodiment of FIG. 24, in accordance with aspects of inventive concepts. In the embodiment shown in FIG. 26, the control button 4 is part of the first leg 42. In alternative embodiments, the control button 4 may be in the second leg 44. In still other embodiments, there can be a control button in each leg, and the different control buttons can control different functions of the device 300.

FIG. 27 is another perspective view of the embodiment of FIG. 24, in accordance with aspects of inventive concepts. Again, the second portion 2 is detached from the first portion in this view.

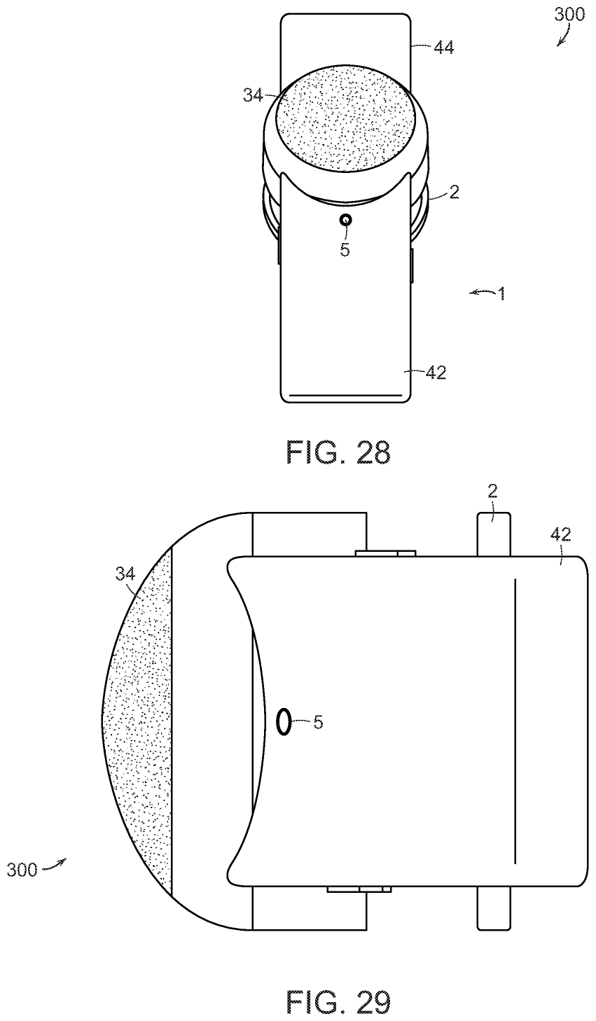

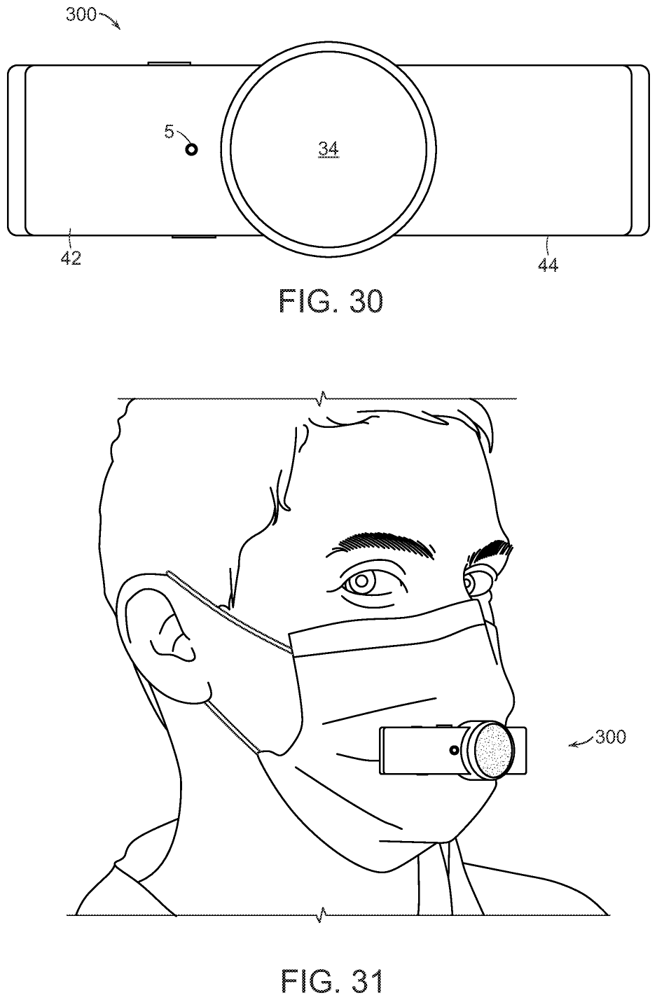

FIG. 28 is a front-perspective view of the embodiment of FIG. 24, in accordance with aspects of inventive concepts. FIG. 29 is a side view of the device 300 of FIG. 24, in accordance with aspects of inventive concepts. FIG. 30 is a rear view of the device 300 of FIG. 24, in accordance with aspects of inventive concepts.

FIG. 31 shows a user wearing the device 300 of FIG. 24, in accordance with aspects of inventive concepts. In this embodiment, the device 300 is coupled to a facemask 30, with the first portion 1 external to the mask and the second portion 2 internal to the mask, as previously described. That is, the second portion 2 can be a ring with magnets that couple to the first portion through the mask 30.

In some embodiments, the removable mask airflow device 300 can comprise a thermometer. In some embodiments, the thermometer can be configured to communicate with a network, e.g., using Bluetooth technology.

Aside from the differences mentioned, the removable air regulator device 300 shown in FIG. 24 may include different characteristics discussed in connection with other embodiments discussed herein.

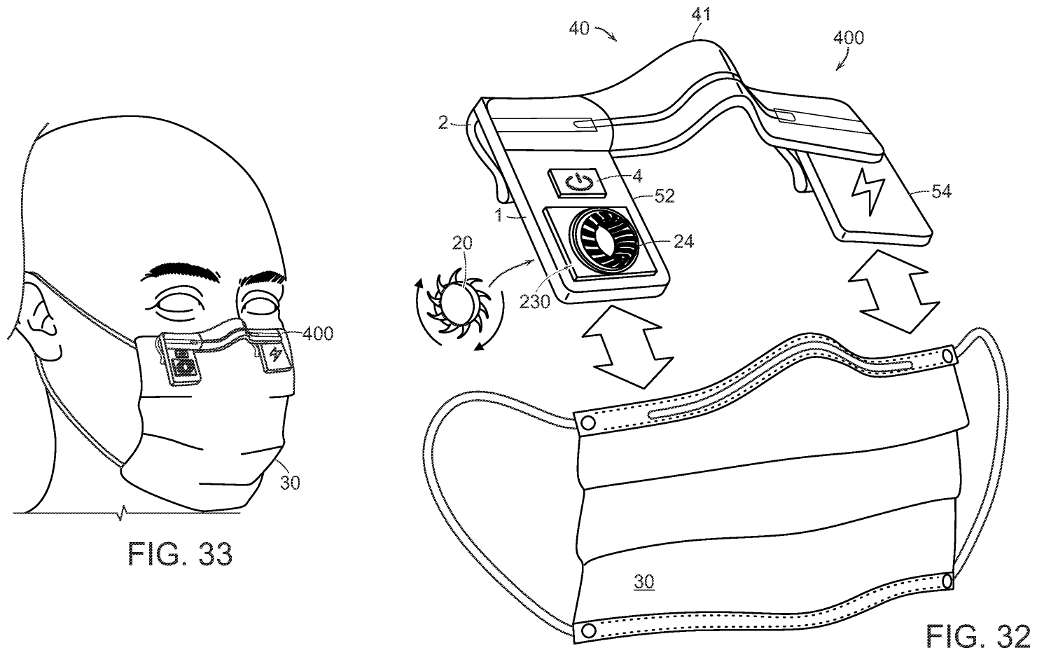

FIG. 32 is a front-perspective view of another alternative embodiment of a removable mask airflow device 400, in accordance with aspects of inventive concepts. Similar to the embodiments previously described, in this embodiment the device 400 comprises a first portion 1 and a second portion 2 with a facemask between the first portion 1 and the second portion 2. Similar to the embodiments, previously described, one or more fan modules (not shown) can be mounted at one or more openings.

In some embodiments, such as the one shown in FIG. 32, the second portion 2 is constructed and arranged as a clip and is integral with the first portion 1. In some embodiments, such as the one shown in FIG. 32, when the device 400 is coupled to a mask 30, the top of the mask is positioned between the second portion 2 and the first portion 1. In this embodiment, the first and second portions combine to form one or more clips that slidingly receive the top portion of the mask.

In some embodiments, such as the one shown in FIG. 32, the first portion 1 of the removable air regulator device 400 comprises one or more legs coupled to a central region 40. In the embodiment shown in FIG. 32, the first portion 1 comprises two legs 52, 54. In alternative embodiments, the first portion 1 can comprise a different number of legs. In this embodiment, the legs are the same or substantially the same length. In alternative embodiments, one or more of the legs can have a different length than one or more of the other legs. In the present embodiment, the legs have the same or substantially the same width. In alternative embodiments, the legs can have different widths.

In some embodiments, such as the one shown in FIG. 32, at least one leg can include at least one fan module. In this embodiment, the first leg 52 comprises at least one fan module 20. In alternative embodiments, the one or more fan modules 20 is located at a different position. In some embodiments, each leg can comprise at least one fan module. In such embodiments, the fans can be independently controlled or jointly controlled. In some embodiments where each leg includes a fan module 20, the fans in the different legs can direct flow the same or differently. For example, in some embodiments, a fan in one leg can direct flow into the mask and a fan in the other leg can direct flow out of the mask.

In some embodiments, such as the one shown in FIG. 32, the central region 40 comprises a nose strap 41 configured to fit at a user's nose. In some embodiments, such as the one shown in FIG. 32, the nose strap 41 is adjustable to conform to the nose of the user. In some embodiments, the nose strap 41 can comprise a memory wire. In some embodiments, the memory wire can be embedded internally in the nose strap 40 to ensure comfort. In some embodiments, the nose strap 41 comprises silicone, and a memory wire can be disposed within the silicone. In alternative embodiments, the nose strap can be formed of and/or comprise different materials that achieve substantially the same physical functions.

In some embodiments, such as the embodiment shown in FIG. 32, the device 400 comprises battery, such as a lithium-ion polymer battery. In alternative embodiments, the device 400 comprises a different type of battery.

In some embodiments, the device 400 is configured in accordance with configurations previously discussed. As examples, the device can include filters and/or emitters as previously described.

FIG. 33 shows a user wearing the device 400 of FIG. 32, in accordance with aspects of inventive concepts.

In some embodiments, the components such as the fan 20, the fan cover 24, and the mesh filter 230 can be easily decoupled from the device and easily cleaned and sanitized. For example, one may disinfect the device under UV light. In other embodiments, one or more of these components can be disposable and replaceable, such as the filter 230.

In some embodiments, the components such as PCB, the battery, and the control device can be arranged in different positions or configurations to achieve a desired balance of weight, or the comfort of the user, or the stable fixation of the device 400 to a mask 30.

In some embodiments, the removable mask airflow device 400 can comprise a thermometer. In some embodiments, the thermometer can be configured to communicate with a network, e.g., using Bluetooth technology.

Aside from the differences mentioned, the removable mask airflow device 400 shown in FIG. 32 may include different characteristics discussed in connection with other embodiments discussed herein.

FIG. 34 shows a perspective view of an embodiment of a round filter 500 configured for a removable mask airflow device, in accordance with aspects of inventive concepts. In the embodiment shown in FIG. 34 the round filter 500 comprises multiple openings, each having a hexagonal shape. Alternative embodiments can have a different number of openings and/or can have one or more openings with a different shape. In various embodiments, the filter can be made of any rigid or semi-rigid material and have an anti-viral and/or anti-bacterial coating.

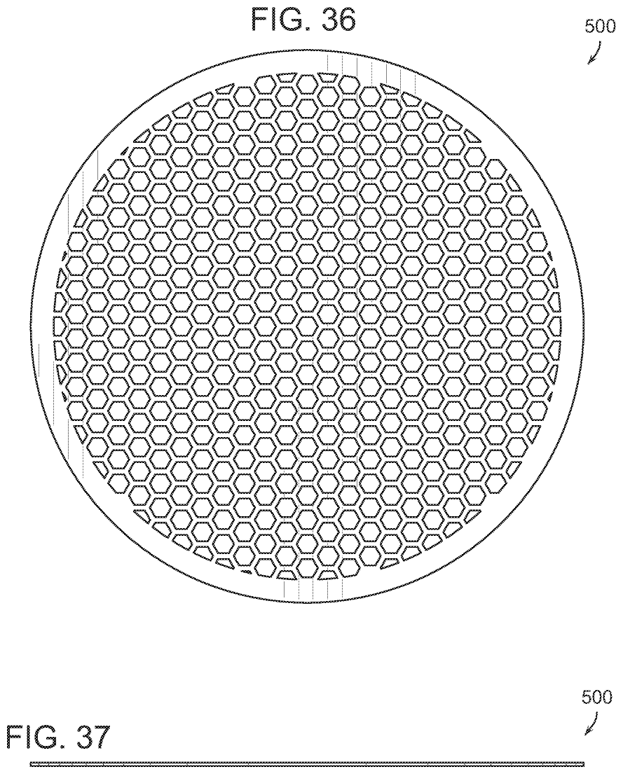

FIG. 35 shows a front view of the embodiment of FIG. 34, in accordance with aspects of inventive concepts. FIG. 36 shows a rear view of the embodiment of FIG. 34, in accordance with aspects of inventive concepts. FIG. 37 shows a side view of the embodiment of FIG. 34, in accordance with aspects of inventive concepts.

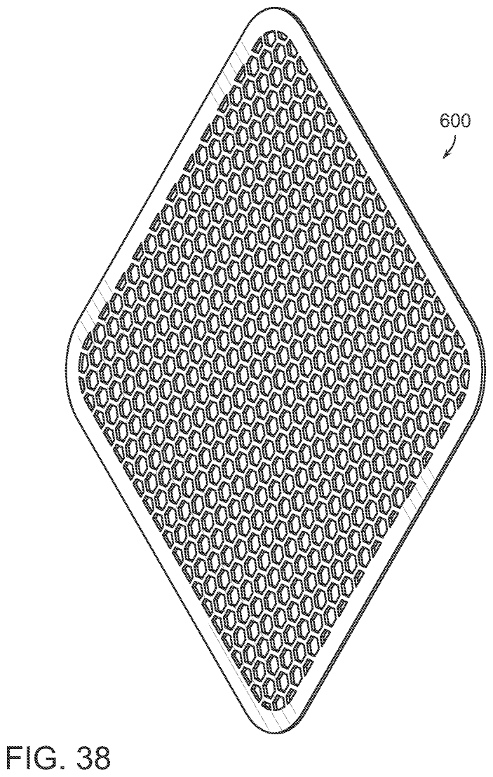

FIG. 38 shows a perspective view of an embodiment of a square filter 600 configured for a removable air regulator device, in accordance with aspects of inventive concepts. In the embodiment shown in FIG. 38 the square filter 600 comprises multiple openings, each having a hexagonal shape. Alternative embodiments can have a different number of openings and/or can have one or more openings with a different shape. In various embodiments, the filter can be made of any rigid or semi-rigid material and have an anti-viral and/or anti-bacterial coating.

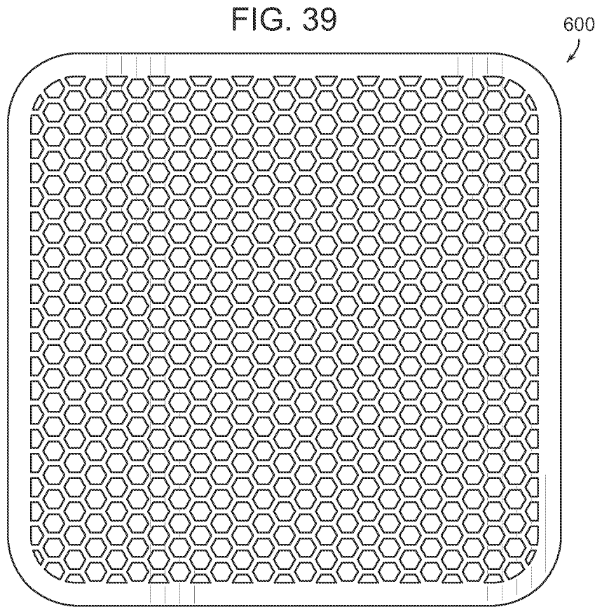

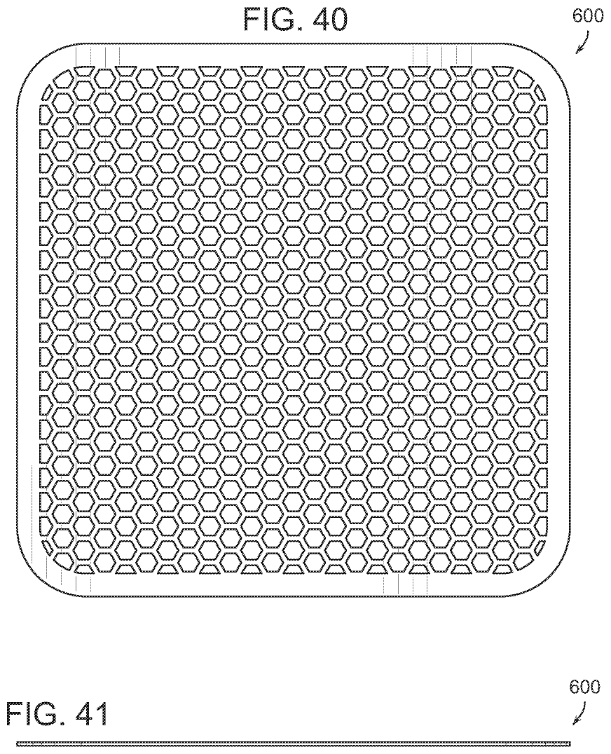

FIG. 39 shows a front view of the embodiment of FIG. 38, in accordance with aspects of inventive concepts. FIG. 40 shows a rear view of the embodiment of FIG. 38, in accordance with aspects of inventive concepts. FIG. 41 shows a side view of the embodiment of FIG. 38, in accordance with aspects of inventive concepts.

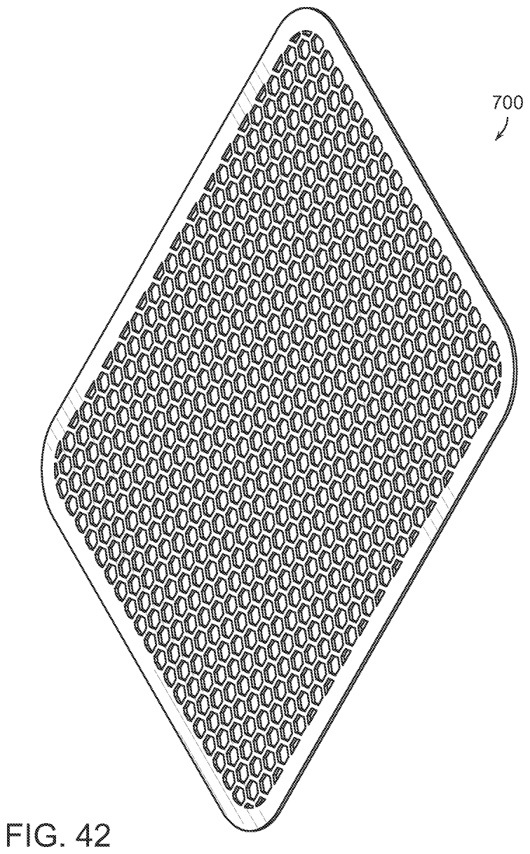

FIG. 42 shows a perspective view of an embodiment of a rectangular filter 700 configured for a removable air regulator device, in accordance with aspects of inventive concepts. In the embodiment shown in FIG. 42 the rectangular filter 700 comprises multiple openings, each having a hexagonal shape. Alternative embodiments can have a different number of openings and/or can have one or more openings with a different shape. In various embodiments, the filter can be made of any rigid or semi-rigid material and have an anti-viral and/or anti-bacterial coating.

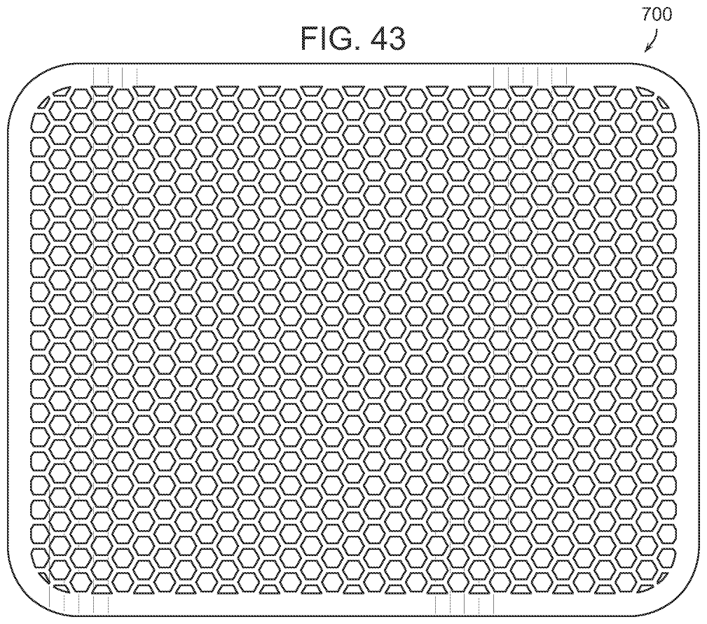

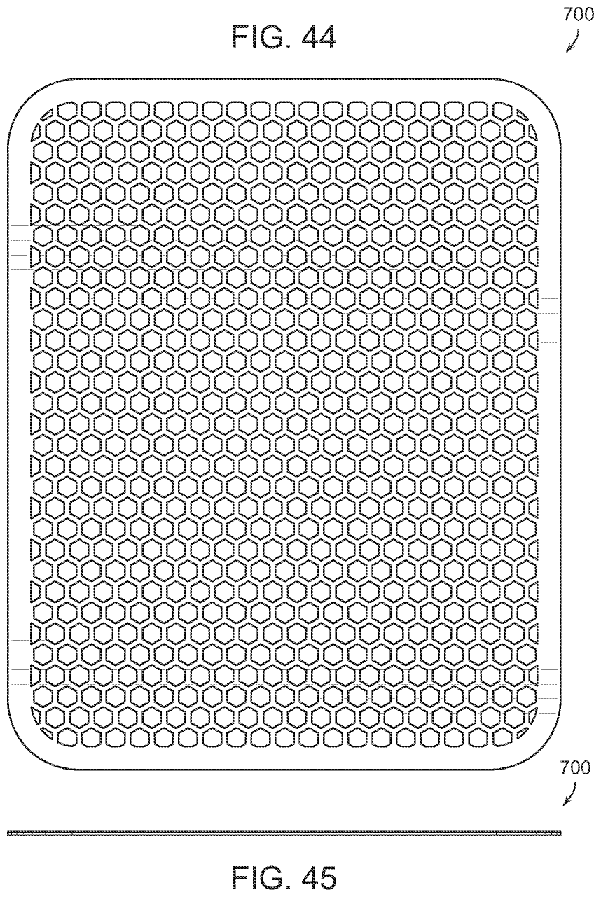

FIG. 43 shows a front view of the embodiment of FIG. 42, in accordance with aspects of inventive concepts. FIG. 44 shows a rear view of the embodiment of FIG. 42, in accordance with aspects of inventive concepts. FIG. 45 shows a side view of the embodiment of FIG. 42, in accordance with aspects of inventive concepts.

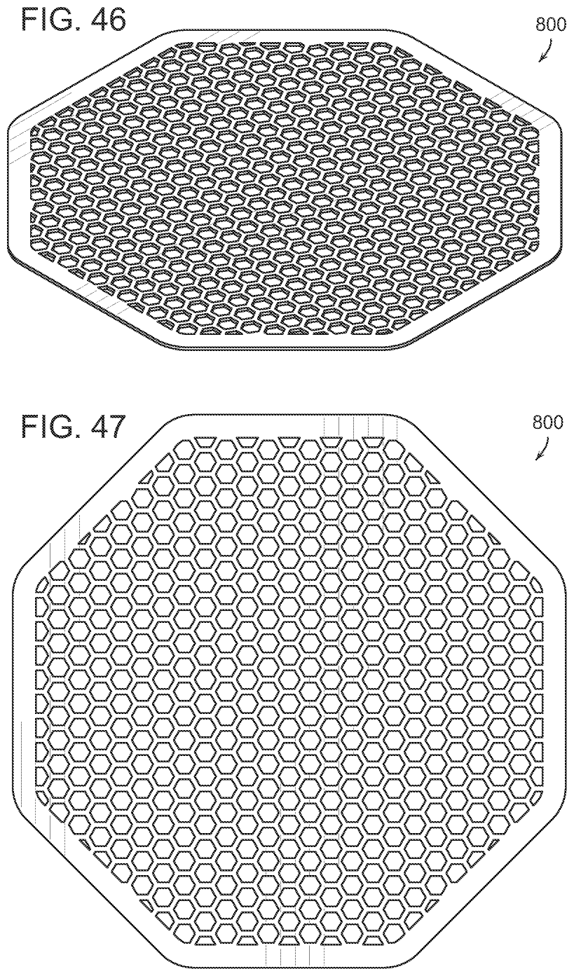

FIG. 46 shows a perspective view of an embodiment of an octagon filter 800 configured for a removable air regulator device, in accordance with aspects of inventive concepts. In the embodiment shown in FIG. 46 the octagon filter 800 comprises multiple openings, each having a hexagonal shape. Alternative embodiments can have a different number of openings and/or can have one or more openings with a different shape. In various embodiments, the filter can be made of any rigid or semi-rigid material and have an anti-viral and/or anti-bacterial coating.

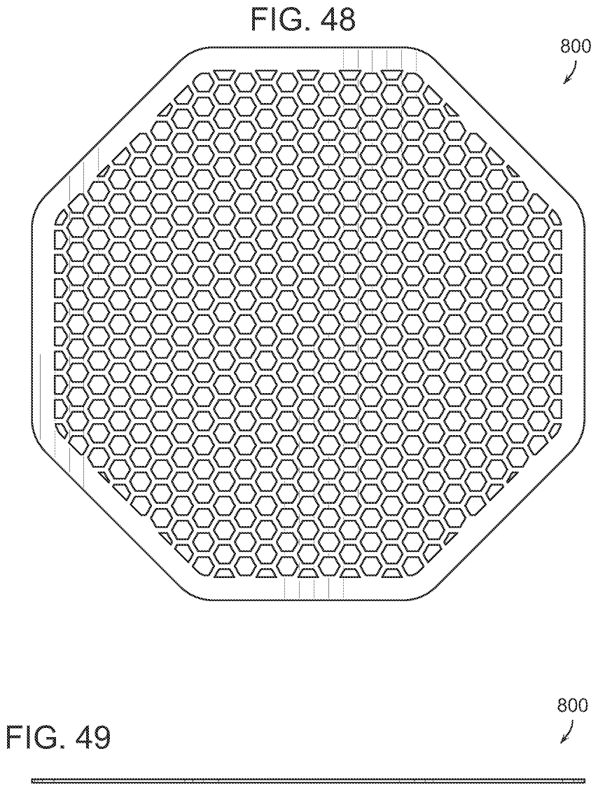

FIG. 47 shows a front view of the embodiment of FIG. 46, in accordance with aspects of inventive concepts. FIG. 48 shows a rear view of the embodiment of FIG. 46, in accordance with aspects of inventive concepts. FIG. 49 shows a side view of the embodiment of FIG. 46, in accordance with aspects of inventive concepts.

While the foregoing has described what are considered to be the best mode and/or other preferred embodiments, it is understood that various modifications can be made therein and that the invention or inventions may be implemented in various forms and embodiments, and that they may be applied in numerous applications, only some of which have been described herein. It is intended by the following claims to claim that which is literally described and all equivalents thereto, including all modifications and variations that fall within the scope of each claim.

It is appreciated that certain features of the invention, which are, for clarity, described in the context of separate embodiments, may also be provided in combination in a single embodiment. Conversely, various features of the invention which are, for brevity, described in the context of a single embodiment may also be provided separately or in any suitable sub-combination. For example, it will be appreciated that all of the features set out in any of the claims (whether independent or dependent) can combined in any given way.

1. A removable airflow device, comprising:
a first portion constructed and arranged to be positioned at an exterior surface of a facemask, the first portion comprising an opening and at least one flow processing device disposed in the opening, the at least one flow processing device configured to process fluid flow through the opening; and
a second portion constructed and arranged to be positioned at an interior surface of the facemask,
wherein the second portion is constructed and arranged to couple with the first portion with a portion of the facemask being between the first portion and the second portion.

2. The device of claim 1, wherein the second portion is removably coupled to the first portion.

3. The device of claim 1, wherein the second portion includes at least one magnetic device configured to magnetically couple to the first portion.

4. The device of claim 1, wherein the at least one flow processing device comprises an ultraviolet light source, the ultraviolet light source oriented to degrade or destroy bacteria and/or viruses passing through the opening.

5. The device of claim 1, wherein the flow processing device comprises a fan disposed within or proximate the opening of the first portion.

6. The device of claim 5, wherein the fan is a variable speed fan.

7. The device of claim 1, wherein the flow processing device comprises a filter disposed at the opening.

8. The device of claim 1, further comprising at least one control mechanism and power source configured to operatively control the at least one flow processing device.

9. The device of claim 1, wherein the at least one flow processing device is a plurality flow processing devices, wherein the plurality flow processing devices comprises at least two different types of flow processing devices selected from a group consisting of a fan, a filter, and an ultraviolet light emitter.

10. The device of claim 1, further comprising a connector for further securing the removable mask airflow device on the facemask.

11. A removable mask airflow device, comprising:
a first portion constructed and arranged to be positioned at an exterior surface of a facemask;
a second portion constructed and arranged to be positioned at an interior surface of the facemask; and
at least one flow processing device having an opening and disposed within the first and/or second portions, the at least one flow processing device configured to process fluid flow through the opening,
wherein the first and second portions form at least one clip constructed and arranged to receive the facemask.

12. The device of claim 11, wherein the first portion comprises a first leg, a second leg, and a central portion between the first and second legs, wherein the central portion is configured to fit at a user's nose.

13. The device of claim 12, wherein the first portion further comprises at least one flow processing device in the first or second leg that is selected from a group consisting of a fan, a filter, and an ultraviolet light emitter.

14. The device of claim 11, wherein the at least one flow processing device comprises an ultraviolet light source, the ultraviolet light source oriented to degrade or destroy bacteria and/or viruses passing through the opening.

15. The device of claim 11, wherein the at least one flow processing further comprises a fan disposed within or proximate the opening.

16. The device of claim 15, wherein the fan is a variable speed fan.

17. The device of claim 15, wherein the at least one flow processing further comprises a filter disposed at the opening.

18. The device of claim 11, further comprising at least one control mechanism and a power source configured to operatively control the at least one flow processing device.

19. The device of claim 11, wherein the at least one flow processing device is a plurality flow processing devices, and
wherein the plurality flow processing devices comprises at least two different types of flow processing devices selected from a group consisting of a fan, a filter, and an ultraviolet light emitter.

20. The device of claim 12, wherein the first leg and the second leg each include at least one of the flow processing devices.
